(19) United States
(12) Patent Application Publication
(10) Pub. No.: US 2009/0191180 A1
(43) Pub. Date: Jul. 30, 2009

(54) Use of Factor VIIa Analogues with Increased Activity

(75) Inventors: Dorthe Viuff, Frederikssund (DK); Mirella Ezban, Copenhagen O (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk HealthCare A/G, Zurich (CH)

(21) Appl. No.: 12/354,509

(22) Filed: Jan. 15, 2009

Related U.S. Application Data
(63) Continuation-in-part of application No. PCT/EP2007/057246, filed on Jul. 13, 2007.
(60) Provisional application No. 60/835,361, filed on Aug. 3, 2006.
(60) Provisional application No. 60/835,356, filed on Aug. 3, 2006.

(30) Foreign Application Priority Data
Jul. 17, 2006 (EP) 06117283.9
Jul. 17, 2006 (EP) 06117284.7
Jul. 6, 2007 (EP) 07111940.8

(51) Int. Cl. A61K 38/36; A61K 38/37; A61K 38/48; A61K 38/55

(52) U.S. Cl. 424/94.64; 514/2; 514/12

(57) ABSTRACT

The invention relates to methods for treatment of bleeding episodes in a subject with thrombocytopenia.

### CROSS-REFERENCE TO RELATED APPLICATIONS

This application is a Continuation-in-Part under 35 U.S.C. 111(a) (i.e. claims benefit under 35 U.S.C. 120) of International Patent Application PCT/EP2007/057246 (published as WO 2008/009635), filed Jul. 13, 2007, and claims priority under 35 U.S.C. § 119 of European Patent Applications 06117283.9, filed Jul. 17, 2006, and 06117284.7, filed Jul. 17, 2006 and 07111940.8, filed Jul. 6, 2007; this application further claims priority of U.S. Provisional Applications 60/835,361 and 60/835,356, filed Aug. 3, 2006.

### FIELD OF THE INVENTION

The present invention relates to methods for treatment of bleeding episodes in a subject with thrombocytopenia, including the prevention of, or minimizing severity of, late complications in bleeding episodes in such subjects with thrombocytopenia.

### BACKGROUND OF THE INVENTION

Haemostasis is a complex physiological process which ultimately results in the arrest of bleeding. This is dependent on the proper function of three main components: blood vessels (especially the endothelial lining), coagulation factors, and platelets. Once a haemostatic plug is formed, the timely activation of the fibrinolytic system is equally important to prevent further unnecessary haemostatic activation. Any malfunction of this system (due to a reduced number, or molecular dysfunction, of the haemostatic components or increased activation of the fibrinolytic components) may lead to clinical bleeding such as, e.g., haemorrhagic diathesis of varying severity.

In most physiological situations, haemostasis is triggered by the interaction of circulating activated coagulation factor VII (FVIIa) with tissue factor (TF) subsequent to exposure of TF at the site of an injury. Endogenous FVIIa becomes proteolytically active only after forming a complex with TF. Normally, TF is expressed in the deep layers of the vessel wall and is exposed following injury. This ensures a highly localized activation of coagulation and prevents disseminated coagulation. TF also seems to exist in a non-active form, so-called encrypted TF. The regulation of encrypted versus active TF is still unknown.

Activated recombinant human factor VII (rFVIIa) is indicated for the treatment of bleeding episodes in haemophilia A or B patients with inhibitors to Factor VIII or Factor IX. When given in high (pharmacological) doses, rFVIIa can bind independently of TF to activated platelets and initiate local thrombin generation which is important for the formation of the initial haemostatic plug.

Thrombocytopenia is the term for a reduced platelet (thrombocyte) count. It occurs when platelets are lost from the circulation faster than they can be replaced from the bone marrow where they are made. The thrombocytes are essential in haemostasis and these conditions of thrombocytopenia are therefore sometimes associated with abnormal serious bleeding.

The platelet count in the circulating blood is normally between 150 and 400×10⁹ per liter of blood, 150-400×10⁹ platelets/L.

Treatment of thrombocytopenia with recombinant wild type FVIIa has earlier been shown to have poor effect when the level of platelets was less than 50×10⁹ platelets/L. Pihusch, M. et al., 2005 Thromb Haemostasis, 3 (9):1935-1944 was not able to show an overall effect of recombinant wild type FVIIa treatment on change in bleeding score.

Thus, there is a need in the art for improved methods and compositions for treatment of thrombocytopenia, as well as for prevention and attenuation of late complications that result from thrombocytopenia.

### SUMMARY OF THE INVENTION

The invention provides the use of a Factor VII polypeptide having increased activity compared to wild-type Factor VIIa, such as V158D/E296V/M298Q-FVIIa for the manufacture of a medicament for treatment of bleeding episodes in a subject with thrombocytopenia. Typical subjects for whom the medicament is used are those subjects who have a very low level of platelets.

The invention also provides methods for preventing or attenuating the symptoms of bleeding episodes in a subject with thrombocytopenia, which are carried out by administering to a subject an effective amount for said preventing or attenuating of a Factor VII polypeptide having increased activity compared to wild-type Factor VIIa, such as V158D/E296V/M298Q-FVIIa.

In some embodiments, the effective amount comprises at least about 1 μg/kg of a Factor VII polypeptide, such as at least about 10 μg/kg, such as at least about 20 μg/kg, such as at least about 40 μg/kg, such as at least about 80 μg/kg of a Factor VII polypeptide having increased activity compared to wild-type Factor VIIa. In some embodiments, the effective amount comprises at least about 100 μg/kg of a Factor VII polypeptide having increased activity compared to wild-type Factor VIIa. In some embodiments, a first amount of at least about 1 μg/kg of a Factor VII polypeptide, such as at least about 10 μg/kg, such as at least about 20 μg/kg, such as at least about 40 μg/kg, such as at least about 80 μg/kg Factor VII polypeptide having increased activity compared to wild-type Factor VIIa is administered at the start of treatment, and a second amount of at least about 1 μg/kg of a Factor VII polypeptide, such as at least about 10 μg/kg, such as at least about 20 μg/kg, such as at least about 40 μg/kg, such as at least about 80 μg/kg of Factor VII polypeptide having increased activity compared to wild-type Factor VIIa is administered to the subject one or more hours after the start of treatment. In some embodiments, a third amount of at least about 1 μg/kg of a Factor VII polypeptide, such as at least about 10 μg/kg, such as at least about 20 μg/kg, such as at least about 40 μg/kg, such as at least about 80 μg/kg of Factor VII polypeptide having increased activity compared to wild-type Factor VIIa is administered at a later time, such as, e.g. least about one hour after the start of the second treatment. It is to be understood that the exact amount of FVII polypeptide administered will vary depending on the specific increase in activity of the Factor VII polypeptide having increased activity compared to wild-type Factor VIIa and also depending on specific thrombocytopenia indication being treated.

In some embodiments, the method further comprises administering to the subject a second coagulation agent in an amount that augments the treatment by said Factor VII polypeptide having increased activity compared to wild-type Factor VIIa, such as V158D/E296V/M298Q-FVIIa. Preferably, the second coagulation agent is a coagulation factor (including, without limitation, Factor V, Factor VIII, Factor IX, Factor X, Factor XI, Factor XIII, Fibrinogen, thrombin, TAFI; an antifibrinolytics such as, e.g., PAI-1, aprotinin, epsilon-aminocaproic acid or tranexamic acid, various antithrombotic treatments, as well as transfusions with platelet, RBC, FFP, oxygen carriers, the various bypassing agents and fluid therapies (colloids/crystalloids), or any combination thereof.

The present invention also provides a kit of parts for treatment of bleeding episodes in subjects with thrombocytopenia, comprising

(i) A medicament comprising a Factor VII polypeptide having increased activity compared to wild-type Factor VIIa; and
(ii) Instructions for use describing that:
a. A first dose containing at least about 1 μg/kg of a Factor VII polypeptide, such as at least about 10 μg/kg, such as at least about 20 μg/kg, such as at least about 40 μg/kg, such as at least about 80, such as at least about 100 μg/kg Factor VII polypeptide having increased activity compared to wild-type Factor VIIa, should be administered at the start of treatment;
b. Optionally, a second dose containing at least about 1 μg/kg of a Factor VII polypeptide, such as at least about 10 μg/kg, such as at least about 20 μg/kg, such as at least about 40 μg/kg, such as at least about 80 μg/kg Factor VII polypeptide having increased activity compared to wild-type Factor VIIa should be administered one to 24 hours after the start of treatment.

The present invention also provides method for treating bleeding episodes in a subject with thrombocytopenia in a majority of subjects with thrombocytopenia, said method comprising (i) administering to a group of subjects with thrombocytopenia having a bleeding an effective amount for said treatment of Factor VII polypeptide having increased activity compared to wild-type Factor VIIa; and (ii) observing a reduction in one or more clinical parameters of said bleeding episode among said group of subjects relative to the level of said clinical parameters that would have been expected in the same group of subjects who had not received said Factor VII polypeptide having increased activity compared to wild-type Factor VIIa.

### DETAILED DESCRIPTION OF THE INVENTION

Thrombocytopenia is defined as a reduced platelet count and is often divided into three major classes:
1) Low production of platelets in the bone marrow;
2) Increased breakdown of platelets in the bloodstream (intravascular) or in the spleen or liver (extravascular); and
3) Dilution.
   Typical disorders that involve low production in the bone marrow include:

a) Certain anemias, such as aplastic anemia;

b) Leukemia;

c) Cancer in the bone marrow;

d) Infections affecting the bone marrow; and

e) Alcohol-induced thrombocytopenia.

Typical disorders that involve the breakdown of platelets include:

a) Immune thrombocytopenic purpura (ITP);

b) Drug-induced immune thrombocytopenia (caused e.g. by heparin);

c) Drug-induced nonimmune thrombocyopenia (caused by e.g. anticancer agents);

d) Thrombotic thrombocytopenic purpura;

e) Transfusion-induced thrombocytopenia;

f) Primary thrombocythemia;

g) Disseminated intravascular coagulation (DIC);

h) Hypersplenism (e.g. cirrhosis);

i) Hemolytic uremic syndrome;

j) Paroxysmal nocturnal hemoglobinuria; and

k) Immune thrombocytopenia (such as thrombocytopenia in LED or RA).

Typical disorders that involve dilution of platelets include:

a) Cardiopulmonary bypass; and

b) Massive RBC transfusion or fluid therapy.

In a first aspect the present invention relates to the use of a Factor VII polypeptide having increased activity compared to wild-type Factor VIIa for the manufacture of a medicament for treating bleeding episodes in a subject with thrombocytopenia.

In a second aspect the present invention relates to a kit of parts for treatment of bleeding episodes in a subject with thrombocytopenia, comprising

(i) A medicament comprising a Factor VII polypeptide having increased activity compared to wild-type Factor VIIa; and
(ii) Instructions for Use describing that:
a. A first dose containing at least about 1 μg/kg of a Factor VII polypeptide, such as at least about 10 μg/kg, such as at least about 20 μg/kg, such as at least about 40 μg/kg, such as at least about 80, such as at least about 100 μg/kg Factor VII polypeptide having increased activity compared to wild-type Factor VIIa, should be administered at the start of treatment;
b. Optionally, a second dose containing at least about 1 μg/kg of a Factor VII polypeptide, such as at least about 10 μg/kg, such as at least about 20 μg/kg, such as at least about 40 μg/kg, such as at least about 80 μg/kg Factor VII polypeptide having increased activity compared to wild-type Factor VIIa should be administered one to 24 hours after the start of treatment.

In a third aspect the present invention relates to a method for treating bleeding episodes in a subject with thrombocytopenia, the method comprising administering to a subject in need of said treatment an effective amount for said treatment of a Factor VII polypeptide having increased activity compared to wild-type Factor VIIa.

In a further aspect the present invention relates to a method for preventing treating bleeding episodes in a subject with thrombocytopenia, the method comprising intentionally administering to a subject in need of said treatment an effective amount for said treatment of Factor VII polypeptide having increased activity compared to wild-type Factor VIIa for the purpose of treating bleeding episodes in a subject with thrombocytopenia.

In a further aspect the present invention relates to a method for treating bleeding episodes in a subject with thrombocytopenia in a majority of subjects with thrombocytopenia, said method comprising (i) administering to a group of subjects with thrombocytopenia having a bleeding an effective amount for said treatment of Factor VII polypeptide having increased activity compared to wild-type Factor VIIa; and (ii) observing a reduction in one or more clinical parameters of said bleeding episode among said group of subjects relative to the level of said clinical parameters that would have been expected in the same group of subjects who had not received said Factor VII polypeptide having increased activity compared to wild-type Factor VIIa.

One aspect of the present invention relates to the use of Factor VII polypeptide having increased activity compared to wild-type Factor VIIa in reducing perioperative blood transfusion requirement in subjects with thrombocytopenia undergoing surgery. In one series of embodiments, treatment of subjects according to the invention results in reduction in a perioperative blood transfusion requirement by 10%, such as 20%, such as 40%, such as 60%, such as 80%, such as 100%.

The term “bleeding episodes” is meant to include uncontrolled and excessive bleeding. Bleeding episodes may be a major problem both in connection with surgery and other forms of tissue damage or it may be spontaneous in subjects with thrombocytopenia. Uncontrolled and excessive bleeding may occur in subjects having apart from thrombocytopenia a further coagulation or bleeding disorder. As used herein the term “bleeding disorder” reflects any defect, congenital, acquired or induced, of cellular or molecular origin that is manifested in bleedings. Examples are clotting factor deficiencies (e.g. haemophilia A and B or deficiency of coagulation Factors XI or VII), clotting factor inhibitors, defective platelet function, or von Willebrand's disease.

Excessive bleedings also occur in subjects with a normally functioning blood clotting cascade (no clotting factor deficiencies or -inhibitors against any of the coagulation factors) and may be caused by a defective platelet function, or von Willebrand's disease. In such cases, the bleedings may be likened to those bleedings caused by haemophilia because the haemostatic system, as in haemophilia, lacks or has abnormal essential clotting “compounds” (such as von Willebrand factor protein) that causes major bleedings.

In subjects who experience extensive tissue damage in association with surgery or vast trauma, the normal haemostatic mechanism may be overwhelmed by the demand of immediate haemostasis and they may develop bleeding in spite of a normal haemostatic mechanism. Achieving satisfactory haemostasis also is a problem when bleedings occur in organs such as the brain, inner ear region and eyes with limited possibility for surgical haemostasis. The same problem may arise in the process of taking biopsies from various organs (liver, lung, tumour tissue, gastrointestinal tract) as well as in laparoscopic surgery. Common for all these situations is the difficulty to provide haemostasis by surgical techniques (sutures, clips, etc.) which also is the case when bleeding is diffuse (haemorrhagic gastritis and profuse uterine bleeding). Acute and profuse bleedings may also occur in subjects on anticoagulant therapy in whom a defective haemostasis has been induced by the therapy given. Such subjects may need surgical interventions in case the anticoagulant effect has to be counteracted rapidly. Radical retropubic prostatectomy is a commonly performed procedure for subjects with localized prostate cancer. The operation is frequently complicated by significant and sometimes massive blood loss. The considerable blood loss during prostatectomy is mainly related to the complicated anatomical situation, with various densely vascularized sites that are not easily accessible for surgical haemostasis, and which may result in diffuse bleeding from a large area. Another situation that may cause problems in the case of unsatisfactory haemostasis is when subjects with a normal haemostatic mechanism are given anticoagulant therapy to prevent thromboembolic disease. Such therapy may include heparin, other forms of proteoglycans, warfarin or other forms of vitamin K-antagonists as well as aspirin and other platelet aggregation inhibitors.

There is a particularly high risk of spontaneous bleeding once the platelet count drops below 10 million per ml. Thus, in one embodiment of the invention, the bleeding is spontaneous due to the thrombocytopenia.

In one embodiment, the bleeding is seen on the skin, such as in the form of pin-prick haemorrhages (purpura), or excessive bruises (ecchymoses) following minor trauma.

In one embodiment, the bleeding is mucosal, such as bleeding from the nose and/or the gums.

In one embodiment, the bleeding is vaginal

In one embodiment, the bleeding is from the eye, such as from retina.

In one embodiment, the bleeding is from the urogenital tract

In one embodiment, the bleeding is excessive bleeding following surgery, dental work or trauma.

In one embodiment, the bleeding is intracranial. In one embodiment, the bleeding is gastrointestinal.

In one embodiment of the invention, the bleeding is associated with haemophilia. In another embodiment, the bleeding is associated with haemophilia with acquired inhibitors. In another embodiment, the bleeding is associated with von Willebrand's disease. In another embodiment, the bleeding is associated with severe tissue damage. In another embodiment, the bleeding is associated with severe trauma. In another embodiment, the bleeding is associated with surgery. In another embodiment, the bleeding is associated with laparoscopic surgery. In another embodiment, the bleeding is associated with haemorrhagic gastritis. In another embodiment, the bleeding is profuse uterine bleeding. In another embodiment, the bleeding is occurring in organs with a limited possibility for mechanical haemostasis. In another embodiment, the bleeding is occurring in the brain, inner ear region or eyes. In another embodiment, the bleeding is associated with the process of taking biopsies. In another embodiment, the bleeding is associated with anticoagulant therapy.

In the present context, the term “treatment” is meant to include both prevention of an expected bleeding, such as in surgery, and regulation of an already occurring bleeding, with the purpose of inhibiting or minimising this bleeding. Prophylactic administration of the Factor VII polypeptide having increased activity compared to wild-type Factor VIIa is thus included in the term “treatment”.

The term “subject” as used herein is intended to mean any animal, in particular mammals, such as humans, and may, where appropriate, be used interchangeably with the term “patient”.

The present invention provides methods and compositions that can be used advantageously to treat bleeding episodes in a subject with thrombocytopenia.

The methods are carried out by administering to this subject with thrombocytopenia Factor VII polypeptide having increased activity compared to wild-type Factor VIIa, in a manner that is effective for treatment. A manner effective for treatment may comprise administering a predetermined amount of Factor VII polypeptide having increased activity compared to wild-type Factor VIIa, and/or utilizing a particular dosage regimen, formulation, mode of administration, combination with other treatments, and the like. The efficacy of the methods of the invention in treating bleeding episodes in a subject with thrombocytopenia may be assessed using one or more conventionally used parameters of the immediate consequences of injury and/or late complications. Immediate consequences include, e.g., blood loss and symptoms of shock; while late complications, include, without limitation, Pulmonary embolism (PE), Acute Respiratory Distress Syndrome (ARDS), Disseminated Intravascular Coagulation (DIC), Acute Myocardial Infarction (AMI), Cerebral Thrombosis (CT), Systemic Inflammatory Response Syndrome (SIRS), infections, sepsis, Multiple Organ Failure (MOF), and Acute Lung Injury (ALI), including death caused by one or more of these syndromes.

Thus in some aspects the present invention relates to a method of reducing the risk of immediate consequences of injury and/or late complications the method comprising administering to a subject in need of said treatment an effective amount for said treatment of Factor VII polypeptide having increased activity compared to wild-type Factor VIIa. In one embodiment the present invention relates to a method of reducing the risk of immediate consequences of injury and/or late complications selected from the list consisting of Pulmonary embolism (PE), Acute Respiratory Distress Syndrome (ARDS), Disseminated Intravascular Coagulation (DIC), Acute Myocardial Infarction (AMI), Cerebral Thrombosis (CT), Systemic Inflammatory Response Syndrome (SIRS), infections, sepsis, Multiple Organ Failure (MOF), and Acute Lung Injury (ALI), including death caused by one or more of these syndromes, the method comprising administering to a subject in need of said treatment an effective amount for said treatment of Factor VII polypeptide having increased activity compared to wild-type Factor VIIa. In one embodiment the late complication is Multiple Organ Failure (MOF). In one embodiment the late complication is Acute Respiratory Distress Syndrome (ARDS). In one embodiment the late complication is Acute Lung Injury (ALI). In one embodiment the late complication is sepsis.

Coagulopathy in trauma is multifactorial, encompassing coagulation abnormalities resembling DIC, caused by systemic activation of coagulation and fibrinolysis; excessive fibrinolysis, which can be evident on the first day in some trauma subjects; and dilutional coagulopathy, which is caused by excessive fluid administration. Some fluids such as hydroxyethyl starch (HES) preparations may directly compromise coagulation. Massive transfusion syndrome results in depletion of coagulation factors and impairment of platelet function. Hypothermia causes a slower enzyme activity of the coagulation cascade and dysfunctional platelets. Metabolic abnormalities, such as acidosis, also compromise coagulation especially when associated with hypothermia.

Non-limiting examples of subjects in need of treatment according to the invention include those who exhibit one or more of the following:
Coagulation abnormalities resembling DIC, caused by systemic activation of coagulation and fibrinolysis
Excessive fibrinolysis
Dilutional coagulopathy caused by excessive fluid treatment, including, without limitation, a limited number of platelets and/or an impaired platelet function compared to the platelet count and platelet activity of normal pooled blood
Receipt of hydroxyethyl starch (HES) preparations
Hypothermia, a including having body temperature below about 37° C., such as, e.g., below about 36° C., below about 35° C., or below about 34° C.
At least one indication of metabolic abnormalities, including, without limitation, acidosis having a blood pH below about 7.5, such as, e.g., below about 7.4, below about 7.3, below about 7.2, or below about 7.1.

In one series of embodiments, subjects treated according to the invention are those who require transfusion with whole blood (WB), packed red blood cells (pRBC), or fresh frozen plasma (FFP), such as, e.g., more than about 2 units, 5 units, or more than about 8 units, between the time of their bleeding and the time of administration of Factor VII polypeptide having increased activity compared to wild-type Factor VIIa. A unit of WB typically contains about 450 ml blood and 63 ml of conventional anticoagulant/preservative (having a hematocrit of 36-44%). A unit of pRBC typically contains 200-250 ml of red blood cells, plasma, and conventional anticoagulant/preservative (having a hematocrit of 70-80%).

Factor VII polypeptide having increased activity compared to wild-type Factor VIIa:

In practicing the present invention, any Factor VII polypeptide having increased activity compared to wild-type Factor VIIa may be used that is effective in treating a bleeding episode in a subject with thrombocytopenia.

The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa.

As used herein, “wild type human FVIIa” is a polypeptide having the amino acid sequence disclosed in U.S. Pat. No. 4,784,950.

Factor VII polypeptide having increased activity compared to wild-type Factor VIIa may include, without limitation, Factor VII polypeptides that have either been chemically modified relative to human Factor VIIa and/or contain one or more amino acid sequence alterations relative to human Factor VIIa. Such Factor VII polypeptides may also apart from activity exhibit other different properties relative to human Factor VIIa, including stability, phospholipid binding, and the like. This includes FVII variants, Factor VII-related polypeptides, Factor VII derivatives and Factor VII conjugates exhibiting increased activity relative to wild-type human Factor VIIa.

The term “Factor VII derivative” as used herein, is intended to designate a FVII polypeptides exhibiting increased activity relative to wild-type Factor VII, in which one or more of the amino acids of the parent peptide have been genetically and/or chemically and/or enzymatically modified, e.g. by alkylation, glycosylation, PEGylation, acylation, ester formation or amide formation or the like. This includes but is not limited to PEGylated human Factor VIIa, cysteine-PEGylated human Factor VIIa and variants thereof.

The term “increased activity” refers to FVII polypeptides with i) increased proteolytic activity compared to recombinant wild type human Factor VIIa or ii) to FVII polypeptides with increased TF binding activity compared to recombinant wild type human Factor VIIa or iii) to FVII polypeptides with increased half life in blood plasma compared to recombinant wild type human Factor VIIa. The term “PEGylated human Factor VIIa” means human Factor VIIa, having a PEG molecule conjugated to a human Factor VIIa polypeptide. It is to be understood, that the PEG molecule may be attached to any part of the Factor VIIa polypeptide including any amino acid residue or carbohydrate moiety of the Factor VIIa polypeptide. The term “cysteine-PEGylated human Factor VIIa” means Factor VIIa having a PEG molecule conjugated to a sulfhydryl group of a cysteine introduced in human Factor VIIa.

For purposes of the invention, Factor VIIa proteolytic activity may be quantified by measuring the ability of a preparation to promote blood clotting using Factor VII-deficient plasma and thromboplastin, as described, e.g., in U.S. Pat. No. 5,997,864. In this assay, proteolytic activity is expressed as the reduction in clotting time relative to a control sample and is converted to “Factor VII units” by comparison with a pooled human serum standard containing 1 unit/ml Factor VII activity. Alternatively, Factor VIIa proteolytic activity may be quantified by (i) measuring the ability of Factor VII polypeptide having increased activity compared to wild-type Factor VIIa to produce of Factor Xa in a system comprising TF embedded in a lipid membrane and Factor X. (Persson et al., J. Biol. Chem. 272:19919-19924, 1997); (ii) measuring Factor X hydrolysis in an aqueous system (see “In Vitro Proteolysis Assay”, Example 3 below); (iii) measuring the physical binding of Factor VII polypeptide having increased activity compared to wild-type Factor VIIa to TF using an instrument based on surface plasmon resonance (Persson, FEBS Letts. 413:359-363, 1997) and (iv) measuring hydrolysis of a synthetic substrate by Factor VII polypeptide having increased activity compared to wild-type Factor VIIa (see “In Vitro Hydrolysis Assay”, Example 2 below).

In a further embodiment of the invention, the factor VII polypeptide is a polypeptide, wherein the ratio between the activity of the Factor VII polypeptide and the activity of the wild-type Factor VIIa polypeptide shown in SEQ ID NO:1 is at least about 1.25. In one embodiment the ratio between the activity of the Factor VII polypeptide and the activity of the wild-type Factor VIIa polypeptide shown in SEQ ID NO:1 is at least about 2.0. In a further embodiment the ratio between the activity of the Factor VII polypeptide and the activity of the wild-type Factor VIIa polypeptide shown in SEQ ID NO: 1 is at least about 4.0.

In a further embodiment of the invention, the factor VII polypeptide is a polypeptide, wherein the ratio between the activity of the Factor VII polypeptide and the activity of the wild-type Factor VIIa polypeptide shown in SEQ ID NO:1 is at least about 1.25 when tested in a Factor VIIa activity assay. In one embodiment the ratio between the activity of the Factor VII polypeptide and the activity of the wild-type Factor VIIa polypeptide shown in SEQ ID NO:1 is at least about 2.0 when tested in a Factor VIIa activity assay. In a further embodiment the ratio between the activity of the Factor VII polypeptide and the activity of the wild-type Factor VIIa polypeptide shown in SEQ ID NO:1 is at least about 4.0 when tested in a Factor VIIa activity assay. The Factor VIIa activity may be measured by the assays described in examples 2 or 3.

In a further embodiment of the invention, the factor VII polypeptide is a polypeptide, wherein the ratio between the activity of the Factor VII polypeptide and the activity of the wild-type Factor VIIa polypeptide shown in SEQ ID NO:1 is at least about 1.25 when tested in the “In Vitro Hydrolysis Assay”. In one embodiment the ratio between the activity of the Factor VII polypeptide and the activity of the wild-type Factor VIIa polypeptide shown in SEQ ID NO:1 is at least about 2.0 when tested in the “In Vitro Hydrolysis Assay”. In a further embodiment the ratio between the activity of the Factor VII polypeptide and the activity of the wild-type Factor VIIa polypeptide shown in SEQ ID NO: 1 is at least about 4.0 when tested in the “In Vitro Hydrolysis Assay”.

In a further embodiment of the invention, the factor VII polypeptide is a polypeptide, wherein the ratio between the activity of the Factor VII polypeptide and the activity of the wild-type Factor VIIa polypeptide shown in SEQ ID NO:1 is at least about 1.25 when tested in the “In Vitro Proteolysis Assay”. In one embodiment the ratio between the activity of the Factor VII polypeptide and the activity of the wild-type Factor VIIa polypeptide shown in SEQ ID NO:1 is at least about 2.0 when tested in the “In Vitro Proteolysis Assay”. In a further embodiment the ratio between the activity of the Factor VII polypeptide and the activity of the wild-type Factor VIIa polypeptide shown in SEQ ID NO: 1 is at least about 4.0 when tested in the “In Vitro Proteolysis Assay”. In a further embodiment the ratio between the activity of the Factor VII polypeptide and the activity of the wild-type Factor VIIa polypeptide shown in SEQ ID NO:1 is at least about 8.0 when tested in the “In Vitro Proteolysis Assay”.

Examples of Factor VII polypeptides having increased activity compared to wild-type Factor VIIa, without limitation, wild-type human Factor VIIa, L305V-FVII, L305V/M306D/D309S-FVII, L305I-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A —FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A —FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A —FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, Factor VIIa lacking the Gla domain; and P11Q/K33E-FVII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn, FVII having substitutions, additions or deletions in the amino acid sequence from 304Arg to 329Cys.

Other examples of factor VII equivalents include, without limitation Factor VII equivalents having substantially the same biological activity as wild-type Factor VII including S52A-FVIIa, S60A-FVIIa (Lino et al., Arch. Biochem. Biophys. 352: 182-192, 1998); FVIIa equivalents exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII equivalents as disclosed in WO 02/29025; and FVII equivalents exhibiting increased proteolytic stability as disclosed in WO 02/38162 (Scripps Research Institute); FVII equivalents having a modified Gla-domain and exhibiting an enhanced membrane binding as disclosed in WO 99/20767, WO 00/66753, WO 02/02764, and US patent application 20030211094 (University of Minnesota); and FVII equivalents as disclosed in WO 01/04287, WO 01/58935, WO 03/93465, and US patent application 20030165996 (Maxygen ApS).

Other examples of factor VII equivalents include GlycoPegylated FVII derivatives as disclosed in WO 03/31464 and US Patent applications US 20040043446, US 20040063911, US 20040142856, US 20040137557, and US 20040132640 (Neose Technologies, Inc.).

Non-limiting examples of FVII variants having increased biological activity compared to wild-type FVIIa include FVII variants as disclosed in WO 01/83725, WO 02/22776, WO 02/077218, WO 03/027147, WO 04/029090, WO 05/075635, European patent application with application number 05108713.8 (Novo Nordisk A/S), WO 02/38162 (Scripps Research Institute); and FVIIa variants with enhanced activity as disclosed in JP 2001061479 (Chemo-Sero-Therapeutic Res Inst.).

In one embodiment of the invention, the factor VII polypeptide is K337A-FVII.

In a further embodiment of the invention, the factor VII polypeptide is V158D-FVII.

In a further embodiment of the invention, the factor VII polypeptide is E296V-FVII.

In a further embodiment of the invention, the factor VII polypeptide is M298Q-FVII.

In a further embodiment of the invention, the factor VII polypeptide is V158T-FVII.

In a further embodiment of the invention, the factor VII polypeptide is S314E-FVII.

In a further embodiment of the invention, the factor VII polypeptide is L305V-FVII.

In a further embodiment of the invention, the factor VII polypeptide is L305V/K337A-FVII.

In a further embodiment of the invention, the factor VII polypeptide is L305V/V158D-FVII.

In a further embodiment of the invention, the factor VII polypeptide is L305V/E296V-FVII.

In a further embodiment of the invention, the factor VII polypeptide is L305V/M298Q-FVII.

In a further embodiment of the invention, the factor VII polypeptide is L305V/V158T-FVII.

In a further embodiment of the invention, the factor VII polypeptide is L305V/S314E-FVII.

In a further embodiment of the invention, the factor VII polypeptide is K337A/S314E-FVII.

In a further embodiment of the invention, the factor VII polypeptide is K337A/V158T-FVII.

In a further embodiment of the invention, the factor VII polypeptide is K337A/M298Q-FVII.

In a further embodiment of the invention, the factor VII polypeptide is K337A/E296V-FVII.

In a further embodiment of the invention, the factor VII polypeptide is K337A/V158D-FVII.

In a further embodiment of the invention, the factor VII polypeptide is V158D/S314E-FVII.

In a further embodiment of the invention, the factor VII polypeptide is V158D/M298Q-FVII.

In a further embodiment of the invention, the factor VII polypeptide is V158D/E296V-FVII.

In a further embodiment of the invention, the factor VII polypeptide is V158T/S314E-FVII.

In a further embodiment of the invention, the factor VII polypeptide is V158T/M298Q-FVII.

In a further embodiment of the invention, the factor VII polypeptide is V158T/E296V-FVII.

In a further embodiment of the invention, the factor VII polypeptide is E296V/S314E-FVII.

In a further embodiment of the invention, the factor VII polypeptide is S314E/M298Q-FVII.

In a further embodiment of the invention, the factor VII polypeptide is E296V/M298Q-FVII.

In a further embodiment of the invention, the factor VII polypeptide is L305V/K337A/V158D-FVII.

In a further embodiment of the invention, the factor VII polypeptide is L305V/K337A/E296V-FVII.

In a further embodiment of the invention, the factor VII polypeptide is L305V/K337A/M298Q-FVII.

In a further embodiment of the invention, the factor VII polypeptide is L305V/K337A/V158T-FVII.

In a further embodiment of the invention, the factor VII polypeptide is L305V/K337A/S314E-FVII.

In a further embodiment of the invention, the factor VII polypeptide is L305V/V158D/E296V-FVII.

In a further embodiment of the invention, the factor VII polypeptide is L305V/V158D/M298Q-FVII.

In a further embodiment of the invention, the factor VII polypeptide is L305V/V158D/S314E-FVII.

In a further embodiment of the invention, the factor VII polypeptide is L305V/E296V/M298Q-FVII.

In a further embodiment of the invention, the factor VII polypeptide is L305V/E296V/V158T-FVII.

In a further embodiment of the invention, the factor VII polypeptide is L305V/E296V/S314E-FVII.

In a further embodiment of the invention, the factor VII polypeptide is L305V/M298Q/V158T-FVII.

In a further embodiment of the invention, the factor VII polypeptide is L305V/M298Q/S314E-FVII.

In a further embodiment of the invention, the factor VII polypeptide is L305V/V158T/S314E-FVII.

In a further embodiment of the invention, the factor VII polypeptide is K337A/S314E/V158T-FVII.

In a further embodiment of the invention, the factor VII polypeptide is K337A/S314E/M298Q-FVII.

In a further embodiment of the invention, the factor VII polypeptide is K337A/S314E/E296V-FVII.

In a further embodiment of the invention, the factor VII polypeptide is K337A/S314E/V158D-FVII.

In a further embodiment of the invention, the factor VII polypeptide is K337A/V158T/M298Q-FVII.

In a further embodiment of the invention, the factor VII polypeptide is K337A/V158T/E296V-FVII.

In a further embodiment of the invention, the factor VII polypeptide is K337A/M298Q/E296V-FVII.

In a further embodiment of the invention, the factor VII polypeptide is K337A/M298Q/V158D-FVII.

In a further embodiment of the invention, the factor VII polypeptide is K337A/E296V/V158D-FVII.

In a further embodiment of the invention, the factor VII polypeptide is V158D/S314E/M298Q-FVII.

In a further embodiment of the invention, the factor VII polypeptide is V158D/S314E/E296V-FVII.

In a further embodiment of the invention, the factor VII polypeptide is V158D/M298Q/E296V-FVII.

In a further embodiment of the invention, the factor VII polypeptide is V158T/S314E/E296V-FVII.

In a further embodiment of the invention, the factor VII polypeptide is V158T/S314E/M298Q-FVII.

In a further embodiment of the invention, the factor VII polypeptide is V158T/M298Q/E296V-FVII.

In a further embodiment of the invention, the factor VII polypeptide is E296V/S314E/M298Q-FVII.

In a further embodiment of the invention, the factor VII polypeptide is L305V/M298Q/K337A/S314E-FVII.

In a further embodiment of the invention, the factor VII polypeptide is L305V/E296V/K337A/S314E-FVII.

In a further embodiment of the invention, the factor VII polypeptide is E296V/M298Q/K337A/S314E-FVII.

In a further embodiment of the invention, the factor VII polypeptide is L305V/E296V/M298Q/K337A —FVII.

In a further embodiment of the invention, the factor VII polypeptide is L305V/E296V/M298Q/S314E-FVII.

In a further embodiment of the invention, the factor VII polypeptide is V158D/E296V/M298Q/K337A-FVII.

In a further embodiment of the invention, the factor VII polypeptide is V158D/E296V/M298Q/S314E-FVII.

In a further embodiment of the invention, the factor VII polypeptide is L305V/V158D/K337A/S314E-FVII.

In a further embodiment of the invention, the factor VII polypeptide is V158D/M298Q/K337A/S314E-FVII.

In a further embodiment of the invention, the factor VII polypeptide is V158D/E296V/K337A/S314E-FVII.

In a further embodiment of the invention, the factor VII polypeptide is L305V/V158D/E296V/M298Q-FVII.

In a further embodiment of the invention, the factor VII polypeptide is L305V/V158D/M298Q/K337A-FVII.

In a further embodiment of the invention, the factor VII polypeptide is L305V/V158D/E296V/K337A-FVII.

In a further embodiment of the invention, the factor VII polypeptide is L305V/V158D/M298Q/S314E-FVII.

In a further embodiment of the invention, the factor VII polypeptide is L305V/V158D/E296V/S314E-FVII.

In a further embodiment of the invention, the factor VII polypeptide is V158T/E296V/M298Q/K337A-FVII.

In a further embodiment of the invention, the factor VII polypeptide is V158T/E296V/M298Q/S314E-FVII.

In a further embodiment of the invention, the factor VII polypeptide is L305V/V158T/K337A/S314E-FVII.

In a further embodiment of the invention, the factor VII polypeptide is V158T/M298Q/K337A/S314E-FVII.

In a further embodiment of the invention, the factor VII polypeptide is V158T/E296V/K337A/S314E-FVII.

In a further embodiment of the invention, the factor VII polypeptide is L305V/V158T/E296V/M298Q-FVII.

In a further embodiment of the invention, the factor VII polypeptide is L305V/V158T/M298Q/K337A-FVII.

In a further embodiment of the invention, the factor VII polypeptide is L305V/V158T/E296V/K337A-FVII.

In a further embodiment of the invention, the factor VII polypeptide is L305V/V158T/M298Q/S314E-FVII.

In a further embodiment of the invention, the factor VII polypeptide is L305V/V158T/E296V/S314E-FVII.

In a further embodiment of the invention, the factor VII polypeptide is E296V/M298Q/K337A/V158T/S314E-FVII.

In a further embodiment of the invention, the factor VII polypeptide is V158D/E296V/M298Q/K337A/S314E-FVII.

In a further embodiment of the invention, the factor VII polypeptide is L305V/V158D/E296V/M298Q/S314E-FVII.

In a further embodiment of the invention, the factor VII polypeptide is L305V/E296V/M298Q/V158T/S314E-FVII.

In a further embodiment of the invention, the factor VII polypeptide is L305V/E296V/M298Q/K337A/V158T-FVII.

In a further embodiment of the invention, the factor VII polypeptide is L305V/E296V/K337A/V158T/S314E-FVII.

In a further embodiment of the invention, the factor VII polypeptide is L305V/M298Q/K337A/V158T/S314E-FVII.

In a further embodiment of the invention, the factor VII polypeptide is L305V/V158D/E296V/M298Q/K337A-FVII.

In a further embodiment of the invention, the factor VII polypeptide is L305V/V158D/E296V/K337A/S314E-FVII.

In a further embodiment of the invention, the factor VII polypeptide is L305V/V158D/M298Q/K337A/S314E-FVII.

In a further embodiment of the invention, the factor VII polypeptide is L305V/E296V/M298Q/K337A/V158T/S314E-FVII

In a further embodiment of the invention, the factor VII polypeptide is L305V/V158D/E296V/M298Q/K337A/S314E-FVII

### Preparations and Formulations:

The present invention encompasses therapeutic administration of Factor VII polypeptide having increased activity compared to wild-type Factor VIIas, which is achieved using formulations that comprise Factor VIIa preparations. As used herein, a “Factor VII preparation” refers to a plurality of Factor VIIa polypeptides, including variants and chemically modified forms, that have been separated from the cell in which they were synthesized, whether a cell of origin or a recombinant cell that has been programmed to synthesize Factor VII polypeptide having increased activity compared to wild-type Factor VIIa.

Separation of polypeptides from their cell of origin may be achieved by any method known in the art, including, without limitation, removal of cell culture medium containing the desired product from an adherent cell culture; centrifugation or filtration to remove non-adherent cells; and the like.

Optionally, Factor VII polypeptides may be further purified. Purification may be achieved using any method known in the art, including, without limitation, affinity chromatography, such as, e.g., on an anti-Factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J.-C. Janson and Lars Ryden, editors, VCH Publishers, New York, 1989. Following purification, the preparation preferably contains less than about 10% by weight, more preferably less than about 5% and most preferably less than about 1%, of non-Factor VII proteins derived from the host cell.

Factor VII and Factor VII-related polypeptides may be activated by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, Factor VII may be activated by passing it through an ion-exchange chromatography column, such as Mono Q® (Pharmacia) or the like. The resulting activated Factor VII may then be formulated and administered as described below.

Pharmaceutical compositions or formulations for use in the present invention comprise a Factor VIIa preparation in combination with, preferably dissolved in, a pharmaceutically acceptable carrier, preferably an aqueous carrier or diluent. A variety of aqueous carriers may be used, such as water, buffered water, 0.4% saline, 0.3% glycine and the like. The preparations of the invention can also be formulated into liposome preparations for delivery or targeting to the sites of injury. Liposome preparations are generally described in, e.g., U.S. Pat. Nos. 4,837,028, 4,501,728, and 4,975,282. The compositions may be sterilised by conventional, well-known sterilisation techniques. The resulting aqueous solutions may be packaged for use or filtered under aseptic conditions and lyophilised, the lyophilised preparation being combined with a sterile aqueous solution prior to administration.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc.

### Treatment Regimen:

In practicing the present invention, Factor VII polypeptide having increased activity compared to wild-type Factor VIIa may be administered to a subject as a single dose comprising a single-dose-effective amount for treating the bleeding associated with thrombocytopenia, or in a staged series of doses which together comprise an effective amount for treating the bleeding associated with thrombocytopenia. An effective amount of Factor VII polypeptide having increased activity compared to wild-type Factor VIIa (see below) refers to the amount of Factor VIIa polypeptide which, when administered in a single dose or in the aggregate of multiple doses, or as part of any other type of defined treatment regimen, produces a measurable improvement in at least one clinical parameter associated with the bleeding associated with thrombocytopenia. When Factor VIIa polypeptides with different activity are administered, an effective amount may be determined by comparing the coagulant activity of the new Factor VIIa polypeptides with that of known Factor VIIa polypeptides and adjusting the amount to be administered proportionately to the predetermined effective dose for the known Factor VIIa polypeptides.

Administration of Factor VII polypeptide having increased activity compared to wild-type Factor VIIa according to the present invention is preferably initiated within about 6 hours after occurrence of the bleeding associated thrombocytopenia, such as, e.g., within about 4 hours, within about 2 hours, or within about 1 hour. Alternatively, administration may be initiated at any time before start of surgery in subjects in need of such surgery, such as within about 6 hours before surgery, such as, e.g., within about 4 hours, within about 2 hours, or within about 1 hour before surgery e.g., immediately before surgery.

Administration of a single dose refers to administration of an entire dose of Factor VII polypeptide having increased activity compared to wild-type Factor VIIa as a bolus over a period of less than about 5 minutes. In some embodiments, the administration occurs over a period of less than about 2.5 minutes, and, in some, over less than about 1 min. Typically, a single-dose effective amount comprises at least about 1 μg/kg of a Factor VII polypeptide, such as at least about 10 μg/kg, such as at least about 20 μg/kg, such as at least about 40 μg/kg human Factor VII polypeptide having increased activity compared to wild-type Factor VIIa, such as, at least about 50 μg/kg, 75 μg/kg, or 90 μg/kg, or at least 150 μg/kg Factor VIIa.

In other embodiments, a single-dose effective amount comprises not more than about 1 μg/kg of a Factor VII polypeptide, not more than about 5 μg/kg, such as not more than about 10 μg/kg, such as not more than about 20 μg/kg human Factor VII polypeptide having increased activity compared to wild-type Factor VII, such as, not more than 30 μg/kg, 50 μg/kg, or 75 μg/kg, or not more than 100 μg/kg Factor VIIa.

In some embodiments, following administration of a single dose of Factor VII polypeptide having increased activity compared to wild-type Factor VIIa according to the invention, the subject receives no further Factor VII polypeptide having increased activity compared to wild-type Factor VIIa for an interval of at least about 30 minutes. In some embodiments the post-administration interval is at least about 45 minutes, such as at least about 1 hour, at least about 1.5 hours, or at least about 2 hours.

In other embodiments, the subject receives Factor VII polypeptide having increased activity compared to wild-type Factor VIIa according to the following regimen: (i) The subject receives a first amount of Factor VII polypeptide having increased activity compared to wild-type Factor VIIa comprising at least about 1 μg/kg of a Factor VII polypeptide, such as at least about 10 μg/kg, such as at least about 20 μg/kg, such as at least about 40 μg/kg; (ii) after a period of at least about 30 minutes, a second amount of Factor VII polypeptide having increased activity compared to wild-type Factor VIIa is administered, the amount comprising at least about 1 μg/kg of a Factor VII polypeptide, such as at least about 10 μg/kg, such as at least about 20 μg/kg, such as at least about 40 μg/kg; and (iii) after a period of at least about 30 minutes from administration of the second dose, a third amount of Factor VII polypeptide having increased activity compared to wild-type Factor VIIa is administered, the amount comprising at least about 1 μg/kg of a Factor VII polypeptide, such as at least about 10 μg/kg, such as at least about 20 μg/kg, such as at least about 40 μg/kg. After a period of at least about 30 minutes following the administration of the third amount, the subject may then receive a further (fourth) amount of Factor VII polypeptide having increased activity compared to wild-type Factor VIIa comprising at least about 1 μg/kg of a Factor VII polypeptide, such as at least about 10 μg/kg, such as at least about 20 μg/kg, such as at least about 40 μg/kg.

In other embodiments, the first amount of Factor VII polypeptide having increased activity compared to wild-type Factor VIIa comprises at least about 1 μg/kg of a Factor VII polypeptide, such as at least about 10 μg/kg, such as at least about 20 μg/kg, such as at least about 40 μg/kg, such as at least about 80 μg/kg, such as at least about 100 μg/kg or at least about 150 μg/kg; in other embodiments, the second amount of Factor VII polypeptide having increased activity compared to wild-type Factor VIIa comprises at least about 75 μg/kg, such as at least about 90 μg/kg; in other embodiments, the third (and optionally fourth) amount of a Factor VII polypeptide having increased activity compared to wild-type Factor VIIa comprises at least about 75 μg/kg, such as at least about 90 μg/kg.

In other embodiments, the subject receives Factor VII polypeptide having increased activity compared to wild-type Factor VIIa according to the following regimen: (i) The subject receives a first amount of Factor VII polypeptide having increased activity compared to wild-type Factor VIIa comprising not more than about 1 μg/kg of a Factor VII polypeptide, such as not more than about 10 μg/kg, such as not more than about 20 μg/kg, such as not more than about 40 μg/kg; (ii) after a period of at least about 30 minutes, a second amount of Factor VII polypeptide having increased activity compared to wild-type Factor VIIa is administered, the amount comprising not more than about 1 μg/kg of a Factor VII polypeptide, such as not more than about 10 μg/kg, such as not more than about 20 μg/kg, such as not more than about 40 μg/kg; and (iii) after a period of at least about 30 minutes from administration of the second dose, a third amount of Factor VII polypeptide having increased activity compared to wild-type Factor VIIa is administered, the amount comprising not more than about 1 μg/kg of a Factor VII polypeptide, such as not more than about 10 μg/kg, such as not more than about 20 μg/kg, such as not more than about 40 μg/kg. After a period of at least about 30 minutes following the administration of the third amount, the subject may then receive a further (fourth) amount of Factor VII polypeptide having increased activity compared to wild-type Factor VIIa comprising not more than about 1 μg/kg of a Factor VII polypeptide, such as not more than about 10 μg/kg, such as not more than about 20 μg/kg, such as not more than about 40 μg/kg.

In other embodiments, the first amount of Factor VII polypeptide having increased activity compared to wild-type Factor VIIa comprises not more than about 1 μg/kg of a Factor VII polypeptide, such as not more than about 10 μg/kg, such as not more than about 20 μg/kg, such as not more than about 40 μg/kg, such as not more than about 80 μg/kg, such as not more than about 100 μg/kg or not more than about 150 μg/kg; in other embodiments, the second amount of Factor VII polypeptide having increased activity compared to wild-type Factor VIIa comprises not more than about 75 μg/kg, such as not more than about 90 μg/kg; in other embodiments, the third (and optionally fourth) amount of a Factor VII polypeptide having increased activity compared to wild-type Factor VIIa comprises not more than about 75 μg/kg, such as not more than about 90 μg/kg.

In one embodiment, the first dose comprises about 200 μg/kg, the second dose about 100 μg/kg, and the third (and optionally fourth) dose about 100 μg/kg.

In other embodiments, the subject receives the second amount of a Factor VII polypeptide having increased activity compared to wild-type Factor VIIa after a period of at least about 45 minutes from the first administration, such as at least about 1 hour, at least about 1.5 hours, at least about 2 hours, at least about 2.5 hours, or at least about 3 hours.

In other embodiments, the subject receives the third (and optionally fourth) amount of Factor VII polypeptide having increased activity compared to wild-type Factor VIIa after a period of at least about 45 minutes from the previous administration, such as at least about 1 hour, at least about 1.5 hours, at least about 2 hours, at least about 2.5 hours, or at least about 3 hours.

In one embodiment, the subject receives a first dose comprising about 200 μg/kg; after a period of about 1 hour, the subject receives a second dose comprising about 100 μg/kg, and after a period of about 3 hours from the first dose, the subject receives a third dose comprising about 100 μg/kg.

The following table illustrates different non-limiting embodiments of the invention:

TABLE 1

| Time post-injury or | | | | Time to | | 2^{nd} Dose | | Time to | | 3^{rd} Dose | | Additional |
|---|---|---|---|---|---|---|---|---|---|---|---|---|
| post excision | | 1^{st} Dose | | 2^{nd} dose | | (optional) | | 3^{rd} dose | | (optional) | | Doses |
| 0-≦1 | h | >1 | mcg/kg | 0-1 | h | >1 | mcg/kg | 0-1 | h | >1 | mcg/kg | As needed |
| | | | | 1-2 | h | | | 1-2 | h | | | |
| | | | | ≧3 | h | | | ≧3 | h | | | |
| | | >40 | mcg/kg | 0-1 | h | >40 | mcg/kg | 0-1 | h | >40 | mcg/kg | |
| | | | | 1-2 | h | | | 1-2 | h | | | |
| | | | | ≧3 | h | | | ≧3 | h | | | |
| | | 100 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |
| | | ≧150 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |
| >1-≦3 | h | >1 | mcg/kg | 0-1 | h | >1 | mcg/kg | 0-1 | h | >1 | mcg/kg | As needed |
| | | | | 1-2 | h | | | 1-2 | h | | | |
| | | | | ≧3 | h | | | ≧3 | h | | | |
| | | >40 | mcg/kg | 0-1 | h | >40 | mcg/kg | 0-1 | h | >40 | mcg/kg | |
| | | | | 1-2 | h | | | 1-2 | h | | | |
| | | | | ≧3 | h | | | ≧3 | h | | | |
| | | 100 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |
| | | ≧150 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |
| >3-<6 | h | >1 | mcg/kg | 0-1 | h | >1 | mcg/kg | 0-1 | h | >1 | mcg/kg | As needed |
| | | | | 1-2 | h | | | 1-2 | h | | | |
| | | | | ≧3 | h | | | ≧3 | h | | | |
| | | >50 | mcg/kg | 0-1 | h | >50 | mcg/kg | 0-1 | h | >50 | mcg/kg | |
| | | | | 1-2 | h | | | 1-2 | h | | | |
| | | | | ≧3 | h | | | ≧3 | h | | | |
| | | 100 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |
| | | ≧150 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |
| >6-<12 | h | >1 | mcg/kg | 0-1 | h | >1 | mcg/kg | 0-1 | h | >1 | mcg/kg | As needed |
| | | | | 1-2 | h | | | 1-2 | h | | | |
| | | | | ≧3 | h | | | ≧3 | h | | | |
| | | >50 | mcg/kg | 0-1 | h | >50 | mcg/kg | 0-1 | h | >50 | mcg/kg | |
| | | | | 1-2 | h | | | 1-2 | h | | | |
| | | | | ≧3 | h | | | ≧3 | h | | | |
| | | 100 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |
| | | ≧150 | mcg/kg | 0-1 | h | 50-100 | mcg/kg | 0-1 | h | 50-100 | mcg/kg | |
| | | | | 1-2 | h | | | 1-2 | h | | | |
| | | | | ≧3 | h | | | ≧3 | h | | | |

It will be understood that the effective amount of Factor VII polypeptide having increased activity compared to wild-type Factor VIIa, as well as the overall dosage regimen, may vary according to the subject's haemostatic status, which, in turn, may be reflected in one or more clinical parameters, including, e.g., relative levels of circulating coagulation factors; amount of blood lost; rate of bleeding; hematocrit, and the like. It will be further understood that the effective amount may be determined by those of ordinary skill in the art by routine experimentation, by constructing a matrix of values and testing different points in the matrix.

For example, in one series of embodiments, the invention encompasses (i) administering a first dose of a Factor VII polypeptide having increased activity compared to wild-type Factor VIIa; (ii) assessing the subject's coagulation status after a predetermined time; and (iii) based on the assessment, administering a further dose of Factor VII polypeptide having increased activity compared to wild-type Factor VIIa if necessary. Steps (ii) and (iii) may be repeated until satisfactory hemostasis is achieved.

According to the invention, a Factor VII polypeptide having increased activity compared to wild-type Factor VIIa may be administered by any effective route, including, without limitation, intravenous, intramuscular, subcutaneous, mucosal, and pulmonary routes of administration. Preferably, administration is by an intravenous route.

### Combination Treatments:

The present invention encompasses combined administration of an additional agent in concert with a Factor VII polypeptide having increased activity compared to wild-type Factor VIIa. In some embodiments, the additional agent comprises a coagulant, including, without limitation, a coagulation factor such as, e.g. Factor V (see, e.g., PCT/DK02/00736), Factor VIII, Factor IX (see, e.g., WO 02/062376), Factor X, Factor XI, Factor XIII (see, e.g., WO 01/85198), Fibrinogen, thrombin, TAFI (see, e.g., PCT/DK02/00734), Antifibrinolytics such as, e.g., PAI-1, aprotinin, epsilon-aminocaproic acid or tranexamic acid (see, e.g., PCT/DK02/00735; PCT/DK02/00742; PCT/DK02/00751; PCT/DK02/00752), various anti-thrombotic treatments, as well as transfusions with platelet, RBC, FFP, oxygen carriers, the various bypassing agents and fluid therapies (colloids/crystalloids) or any combination thereof. [Are these also relevant?: inhibitors of tissue factor pathway inhibitor (TFPI inhibitors) (see, e.g., WO 01/85199); protein C inhibitors (see, e.g., PCT/DK02/00737); thrombomodulin (see, e.g., PCT/DK02/00738); protein S inhibitors (see, e.g., PCT/DK02/00739); tissue plasminogen activator inhibitors (see, e.g., PCT/DK02/00740); α2-antiplasmin (see, e.g., PCT/DK02/00741);

It will be understood that, in embodiments comprising administration of combinations of Factor VIIa with other agents, the dosage of Factor VII polypeptide having increased activity compared to wild-type Factor VIIa may on its own comprise an effective amount and additional agent(s) may further augment the therapeutic benefit to the subject. Alternatively, the combination of Factor VIIa or equivalent and the second agent may together comprise an effective amount for treating the bleeding associated with thrombocytopenia. It will also be understood that effective amounts may be defined in the context of particular treatment regimens, including, e.g., timing and number of administrations, modes of administrations, formulations, etc.

The present invention is further illustrated by the following examples which, however, are not to be construed as limiting the scope of protection. The features disclosed in the foregoing description and in the following examples may, both separately and in any combination thereof, be material for realising the invention in diverse forms thereof.

### BRIEF DESCRIPTION OF THE DRAWINGS

FIG. 1 shows the full amino acid sequence of native (wild type) human coagulation Factor VII (SEQ ID NO:1).

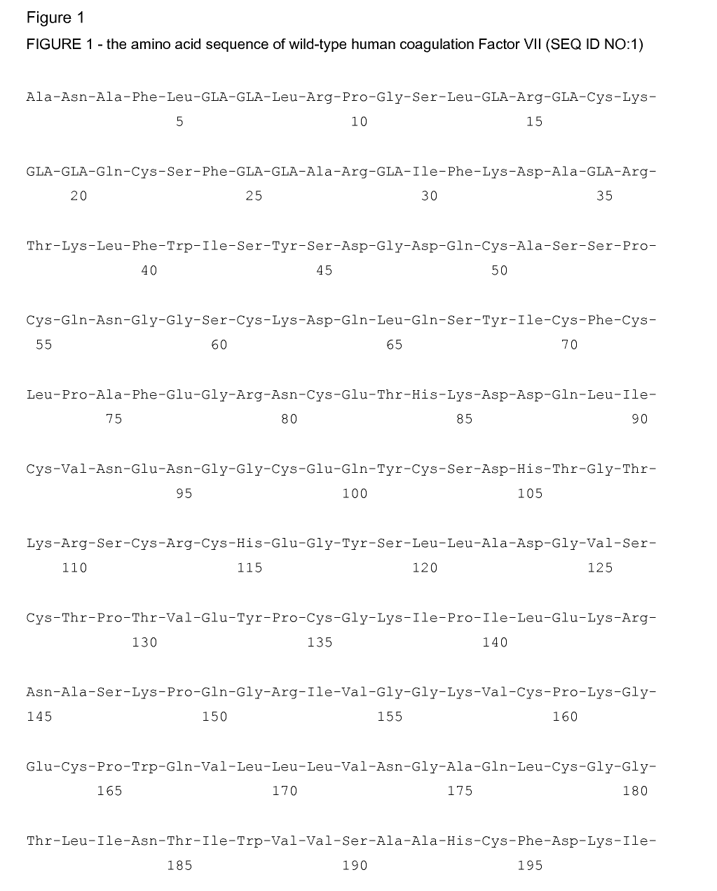

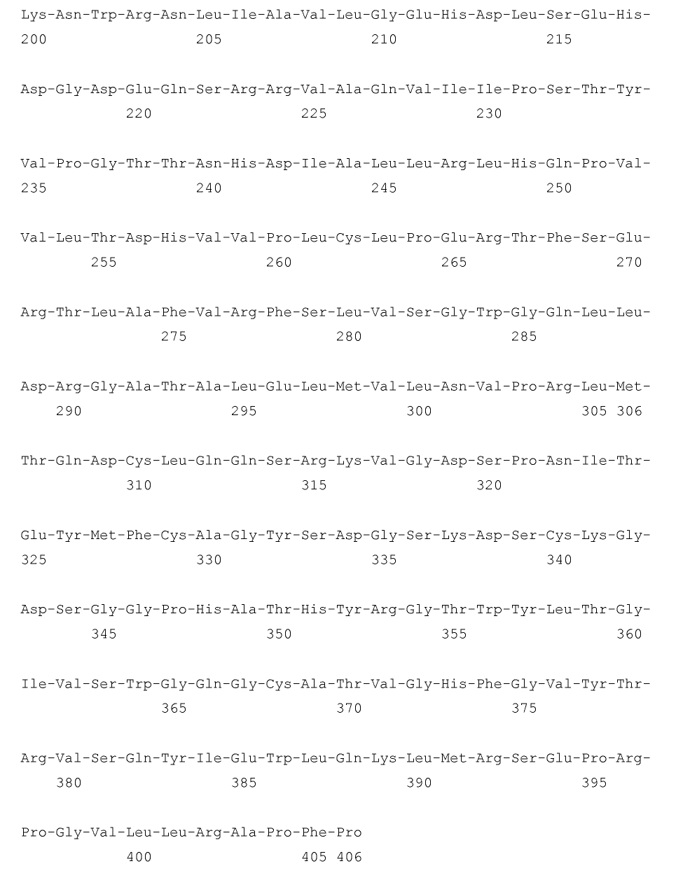

### EXAMPLES

### Example 1

The terminology for amino acid substitutions used the following examples are as follows. The first letter represent the amino acid naturally present at a position of SEQ ID NO:1. The following number represent the position in SEQ ID NO:1. The second letter represent the different amino acid substituting for (replacing) the natural amino acid. An example is M298Q, where an methionine at position 298 of SEQ ID NO:1 is replaced by a glutamine. In another example, V158T/M298Q, the valine in position 158 of SEQ ID NO:1 is replaced by a threonine and the methionine in position 298 of SEQ ID NO:1 is replaced by a Glutamine in the same Factor VII polypeptide.

FVIIa polypeptides having increased activity compared to wild-type Factor VIIa to be used according to the invention may be prepared according to published international patent applications, e.g. WO 01/83725, WO 02/22776, WO 02/077218, WO 03/027147, WO 04/029090, WO 05/075635, European patent application with application number 05108713.8 (Novo Nordisk A/S), WO 02/38162 and JP 2001061479.

### Example 2

### In Vitro Hydrolysis Assay

Native (wild-type) Factor VIIa and Factor VIIa variant (both hereafter referred to as “Factor VIIa”) are assayed in parallel to directly compare their specific activities. The assay is carried out in a microtiter plate (MaxiSorp, Nunc, Denmark). The chromogenic substrate D-Ile-Pro-Arg-p-nitroanilide (S-2288, Chromogenix, Sweden), final concentration 1 mM, is added to Factor VIIa (final concentration 100 nM) in 50 mM Hepes, pH 7.4, containing 0.1 M NaCl, 5 mM CaCl2 and 1 mg/ml bovine serum albumin. The absorbance at 405 nm is measured continuously in a SpectraMax™ 340 plate reader (Molecular Devices, USA). The absorbance developed during a 20-minute incubation, after subtraction of the absorbance in a blank well containing no enzyme, is used to calculate the ratio between the activities of variant and wild-type Factor VIIa:

Ratio=(A405 nm Factor VIIa variant)/(A405 nm Factor VIIa wild-type).

### Example 3

### In Vitro Proteolysis Assay

Native (wild-type) Factor VIIa and Factor VIIa variant (both hereafter referred to as “Factor VIIa”) are assayed in parallel to directly compare their specific activities. The assay is carried out in a microtiter plate (MaxiSorp, Nunc, Denmark). Factor VIIa (10 nM) and Factor X (0.8 microM) in 100 microL 50 mM Hepes, pH 7.4, containing 0.1 M NaCl, 5 mM CaCl2 and 1 mg/ml bovine serum albumin, are incubated for 15 min. Factor X cleavage is then stopped by the addition of 50 microL 50 mM Hepes, pH 7.4, containing 0.1 M NaCl, 20 mM EDTA and 1 mg/ml bovine serum albumin. The amount of Factor Xa generated is measured by addition of the chromogenic substrate Z-D-Arg-Gly-Arg-p-nitroanilide (S-2765, Chromogenix, Sweden), final concentration 0.5 mM. The absorbance at 405 nm is measured continuously in a SpectraMax™ 340 plate reader (Molecular Devices, USA). The absorbance developed during 10 minutes, after subtraction of the absorbance in a blank well containing no FVIIa, is used to calculate the ratio between the proteolytic activities of variant and wild-type Factor VIIa:

Ratio=(A405 nm Factor VIIa variant)/(A405 nm Factor VIIa wild-type).

### Example 4

The effect of wild type recombinant wild type FVIIa (rwtFVIIa) as well as FVIIa analogous with increased activity, such as V158D/E296V/M298Q-FVIIa in blood obtained from stem cell transplantation subjects containing platelets less than 20×109 platelets/L was investigated demonstrating that V158D/E296V/M298Q-FVIIa ex vivo is significantly superior to equimolar concentration of rwtFVIIa in WB obtained from SCT subjects indicating that V158D/E296V/M298Q-FVIIa may have a pronounced effect in spite of a low platelet number.

We investigated by using thromboelastography (TEG 5000 analyser, Haemoscope corporation) the impact of rwtFVIIa (25 nM≈90 ug/kg, 100 nM≈360 ug/kg) and the rFVIIa analogue (25 nM≈90 ug/kg) on whole blood (WB) obtained from seven SCT subjects with platelet counts below 20×10⁹/L. The coagulation was initiated with a low amount of tissue factor (Innovin, final dilution 1:50000) and the clot stability was evaluated by adding tPA (1.8 nM). The clotting time (R-time, sec), the clot formation rate (CFR, α-angle), maximum mechanical strength (MA, m) and the resistance against fibrinolysis determined as the area under the fibrinolysis curve calculated from MA (AUC, mm×sec) were all recorded. Data are presented as mean and the statistical analysis was performed by a two-way ANOVA model. P<0.05 was considered statistically significant.

Only two out of seven subjects formed a clot in the control samples. Five out of seven subjects responded on ex vivo spiking with 25 nM rwtFVIIa whereas effect was observed in all subjects spiked with 100 nm rwtFVIIa and 25 nM V158D/E296V/M298Q-FVIIa, respectively. V158D/E296V/M298Q-FVIIa (25 nM) and rwtFVIIa (25 nM, 100 nM) significantly improved the R-time, MA, and CFR(R: Buffer=2789, rwtFVIIa 25 nM=1713, rwtFVIIa 100 nM=1035, V158D/E296V/M298Q-FVIIa=549; MA: Buffer=0.7, rwtFVIIa 25 nM=3.8, rwtFVIIa 100 nM=7.2, V158D/E296V/M298Q-FVIIa=12.3; CFR: Buffer=1.4, rwtFVIIa 25 nM=5.8, rwtFVIIa 100 nM=10.4, V158D/E296V/M298Q-FVIIa=25.3), except for AUC where the treatment was only significantly different comparing 25 nM rwtFVIIa with 25 nM V158D/E296V/M298Q-FVIIa (AUC: Buffer=239, rwtFVIIa 25 nM=378, rwtFVIIa 100 nM=727, V158D/E296V/M298Q-FVIIa=1031).

However, 25 nM and 100 nM rwtFVIIa were not sufficient to obtained TEG values in the range of normal donors whereas V158D/E296V/M298Q-FVIIa obtained these TEG values in respect to R-time (5 subjects), CFR and MA (3 subjects).

V158D/E296V/M298Q-FVIIa demonstrated significant and superior effect compared to the effect obtained by rwtFVIIa in whole blood from Stem cell transplantation subjects containing platelets less than 20×109 platelets/L. Thus, V158D/E296V/M298Q-FVIIa may be superior to rFVIIa under thrombocytopenia conditions in respect to clot formation and fibrinolysis.

The present data demonstrates that FVIIa analogues with increased activity has a superior impact on hemostasis versus rwtFVIIa on blood obtained from Stem Cell Transplantation (SCT) Subjects having less than 20×109 platelets/L.

### Example 5

### Effect of Wild-Type Human FVIIa and V158D/E296V/M298Q-FVIIa on Tail-Bleeding in Thrombocytopenic Rats

Thrombocytopenia may be caused by a number of underlying diseases, and may be the cause of uncontrolled bleeding. Currently, the standard therapy against thrombocytopenic bleeding is platelet transfusion. The aim of the present study was to examine the effect of wild-type human FVIIa and V158D/E296V/M298Q-FVIIa, an rFVIIa-analogue with increased potency, in rats with antibody-induced thrombocytopenia.

Rats received a subcutaneous injection of polyclonal rabbit anti-rat thrombocyte-antibody

(Accurate Chemicals, Westbury, N.Y.). After 24 hours, when the tail transsection was performed, the platelet number was reduced with more than 90% of the initial value (589±185 to 46±21×10⁹/l; n=58; mean±SD). After 5 minutes of bleeding, rats were treated with wild-type human FVIIa (5 or 10 mg/kg), V158D/E296V/M298Q-FVIIa (10 mg/kg) or vehicle, where after the bleeding was observed for 1800 seconds.

TABLE 2
Total bleeding time and blood loss

| | | Total bleeding | Blood loss |
|---|---|---|---|
| | Group | time (s) | (nmol hemglobin/ml) |
| A | Normal rats (n = 10) | 91 ± 44 | 4.0 ± 4.0 |
| B | Thrombocytopenia + vehicle (n = 12) | 1436 ± 161 *** vs. A | 78.7 ± 22.7 *** vs. A |
| C | Thrombocytop. + 5 mg/kg wild-type | 896 ± 200 | 46.8 ± 24.6 |
| | human FVIIa (n = 12) | | |
| D | Thrombocytop. + 10 mg/kg wild-type | 310 ± 67 ** vs. B | 15.2 ± 9.7 |
| | human FVIIa (n = 11) | | |
| E | Thrombocytop. + 10 mg/kg | 550 ± 207 ** vs. B | 0 ± 0 ^{#} ** vs. B |
| | V158D/E296V/M298Q-FVIIa (n = 11) | | |

Data are mean ± SEM. Bleeding time data are analyzed using Mann-Whitneys U-test (A-B) or Kruskall-Wallis test with Dunn's post-test (B-E). Blood loss data are analyzed after log(x + 1) transformation using Student's t-test (A-B) or one-way ANOVA with Bonferroni's post-test (B-E). Asterisks indicate statistical significance at:
** p < 0.01 and
*** p < 0.001.
^{#} below detection limit.

Wild-type human FVIIa (10 mg/kg) caused a significant reduction in bleeding time, whereas a numerical reduction in blood loss did not reach statistical significance (table 2). V158D/E296V/M298Q-FVIIa reduced both bleeding time and blood loss significantly. In fact, no blood loss, detected as haemoglobin concentration, was detected in V158D/E296V/M298Q-FVIIa treated animals, indicating that the observed bleeding time, was due to oozing of plasma components rather than erythrocytes.

In conclusion, both wild-type human FVIIa and V158D/E296V/M298Q-FVIIa had an effect on tail-bleeding in thrombocytopenic rats with less than 10% of the normal platelet counts. This study indicates that V158D/E296V/M298Q-FVIIa may have a beneficial effect in the treatment of bleeding episodes caused by a severe reduction in the level thrombocytes.

### Example 7

### Effect of Wild-Type Human FVIIa and V158D/E296V/M298Q-FVIIa on Clopidogrel-Induced Bleeding in Rats

Clopidogrel (Plavix; Sanofi Aventis) is an irreversible ADP receptor antagonist, which effectively inhibits platelet aggregation. Clopidogrel is extensively used for prevention of thromboembolic events e.g. myocardial infarctions. One of the potential adverse events following clopidogrel treatment is uncontrolled bleeding, e.g. if acute surgical intervention is needed. Currently, no effective antidote for clopidogrel exists.

The hypothesis of the present study was that wild-type human FVIIa, which currently are registered for use against bleeding in inhibitor-complicated haemophilia, may be used to treat bleeding caused by clopidogrel. Thus, the effect of wild-type human FVIIa and, V158D/E296V/M298Q-FVIIa, a new potent rFVIIa-analogue, was tested in a tail-bleeding model in rats pretreated with clopidogrel.

Rats were dosed orally with 10 mg/kg clopidogrel. After 4 hours, tail-transsection was performed. Five minutes after, the initiation of the bleeding, the rats were treated with wild-type human FVIIa (5, 10, 20 mg/kg), V158D/E296V/M298Q-FVIIa (2, 5, 10 mg/kg) or vehicle, where after the blood loss was determined in the following 30 minutes.

TABLE 4
Blood loss (nmol hemglobin; n = 10)

| | | wild-type | |
|---|---|---|---|
| | Group | human FVIIa | V158D/E296V/M298Q-FVIIa |
| A | Normal rats | 510 ± 465 | |
| B | Clopidogrel + vehicle | 13933 ± 2434 *** vs. A | |
| C | Clopidogrel + 2 mg/kg wild-type | nd | 6703 ± 2097 |
| | human FVIIa/V158D/E296V/M298Q-FVIIa | | |
| D | Clopidogrel + 5 mg/kg wild-type | 8573 ± 2199 ^{£} | 3031 ± 1164 ** vs. B |
| | human FVIIa/V158D/E296V/M298Q-FVIIa | | |
| E | Clopidogrel + 10 mg/kg wild-type | 3867 ± 1468 ** vs. B ^{$} | 1241 ± 1044 *** vs. B ^{$} |
| | human FVIIa/V158D/E296V/M298Q-FVIIa | | |
| F | Clopidogrel + 20 mg/kg wild-type | 3112 ± 678 ** vs. B ^{$} | nd |
| | human FVIIa/V158D/E296V/M298Q-FVIIa | | |

Data are mean ± SEM. Data are analyzed after square root transformation using Student's t-test (A-B) or one-way ANOVA with Bonferroni's post-test (B-F). Asterisks indicate statistical significance at:
** p < 0.01 and
*** p < 0.001. nd: not determined.
^{£}n = 8;
^{$} n = 9

Clopidogrel significantly increased the blood loss compared to normal rats (p<0.001). This increase in blood loss was significantly and dose-dependently reduced by both wild-type human FVIIa and V158D/E296V/M298Q-FVIIa. The dose-response curves were fitted to a sigmoidal curve, showing a higher potency of V158D/E296V/M298Q-FVIIa compared to wild-type human FVIIa. An observed higher maximum effect of V158D/E296V/M298Q-FVIIa compared to wild-type human FVIIa, may indicate a higher efficacy of V158D/E296V/M298Q-FVIIa.

### Example 8

### Effect of Wild-Type Human FVIIa and V158D/E296V/M298Q-FVIIa on Low Molecular Weight Heparin-Induced Bleeding in Rats

The aim of the present experiment was to compare the effect of wild-type human FVIIa and V158D/E296V/M298Q-FVIIa, an rFVIIa-analogue with increased potency, in rats anticoagulated with two different doses of tinzaparin. Thus, rats received 500 or 1800 IU/kg of tinzaparin intravenously. After 10 minutes tail cut was performed, and after another 5 minutes rats were treated with 20 mg/kg wild-type human FVIIa, 10 mg/kg V158D/E296V/M298Q-FVIIa or vehicle, where after the bleeding was observed for 1800 seconds.

TABLE 5
Total bleeding time (s)

| | | Tinzaparin | Tinzaparin |
|---|---|---|---|
| | Group | (500 IU/kg; n = 9) | (1800 IU/kg; n = 10) |
| A | No tinzaparin | 542 ± 180 (n = 9) | |
| B | Tinzaparin + Vehicle | 11800 ± 0 *** vs. A | 1800 ± 0 |
| C | Tinzaparin + 20 mg/kg wild-type | 1038 ± 206 * vs. B | 1357 ± 123 NS vs. B |
| | human FVIIa | | |
| D | Tinzaparin + 10 mg/kg | 404 ± 143 *** vs. B | 463 ± 92 *** vs. B; * vs. C |
| | V158D/E296V/M298Q-FVIIa | | |

Data are mean ± SEM. Data are analyzed using Mann-Whitneys U-test (A-B) or Kruskall-Wallis test with Dunn's post-test (B-D). Asterisks indicate statistical significance at:
* p < 0.05 and
*** p < 0.001.
^{#} observation period 1800 s.

TABLE 6
Blood loss (nmol hemoglobin)

| | | Tinzaparin | Tinzaparin |
|---|---|---|---|
| | Group | (500 IU/kg; n = 9) | (1800 IU/kg; n = 10) |
| A | No tinzaparin | 316 ± 316 (n = 9) | |
| B | Tinzaparin + Vehicle | 9836 ± 2415 *** vs. A | 13179 ± 3048 |
| C | Tinzaparin + 20 mg/kg wild-type | 1470 ± 614 * vs. B | 4383 ± 1050 NS vs. B |
| | human FVIIa | | |
| D | Tinzaparin + 10 mg/kg | 0 ± 0 ^{#} *** vs. B | 351 ± 149 *** vs. B & C |
| | V158D/E296V/M298Q-FVIIa | | |

Data are mean ± SEM. Data are analyzed after log(x + 1) transformation using Student's t-test (A-B) or one-way ANOVA with Bonferroni's post-test (B-E). Asterisks indicate statistical significance at:
* p < 0.05 and
*** p < 0.001.
^{#} below detection limit.
NS: non-significant

wild-type human FVIIa (20 mg/kg) caused a significant reduction in bleeding time and blood loss during a bleeding induced by 500 IU/kg tinzaparin (table 5 and 6, left column), while, V158D/E296V/M298Q-FVIIa was capable to normalize both bleeding time and blood loss.

After severe anticoagulation with 1800 IU/kg tinzaparin, a highly significant effect of 10 mg/kg V158D/E296V/M298Q-FVIIa (10 mg/kg) on bleeding time and blood loss compared to the vehicle control group was retained (table 5 and 6, right column). In contrast, a two-fold higher dose of wild-type human FVIIa (20 mg/kg) did not affect bleeding time or blood loss significantly, although a numerical reduction was observed in both variables. This difference in effect between V158D/E296V/M298Q-FVIIa and wild-type human FVIIa reached statistical significance for bleeding time as well as blood loss.

In conclusion, both wild-type human FVIIa and V158D/E296V/M298Q-FVIIa had an effect on tinzaparin-induced bleeding in rats, but under severely anticoagulated animals V158D/E296V/M298Q-FVIIa seemed more efficacious than wild-type human FVIIa.

### Embodiments of the Invention

1. Use of a Factor VII polypeptide having increased activity compared to wild-type Factor VIIa for the manufacture of a medicament for treating bleeding episodes in a subject with thrombocytopenia.
2. Use according to any of embodiments 1, wherein the ratio between the activity of said Factor VII polypeptide and the activity of the wild-type Factor VIIa polypeptide shown in SEQ ID NO:1 is at least about 1.25.
3. The Factor VII polypeptide according to embodiment 2, wherein said ratio is at least about 2.0, such as at least about 4.0, such as at least about 6, such as at least about 10.
4. Use according to any one of embodiments 1 to 3, wherein the medicament comprises at least about 1 μg/kg of a Factor VII polypeptide, such as at least about 10 μg/kg, such as at least about 20 μg/kg, such as at least about 40 μg/kg, such as at least about 80 μg/kg, such as at least about 100 μg/kg of a Factor VII polypeptide having increased activity compared to wild-type Factor VIIa.
5. Use according to any one of embodiments 1 to 4, wherein the medicament is for administration in a first dose containing at least about 1 μg/kg of a Factor VII polypeptide, such as at least about 10 μg/kg, such as at least about 20 μg/kg, such as at least about 40 μg/kg, such as at least about 80 μg/kg of a Factor VII polypeptide having increased activity compared to wild-type Factor VIIa, followed by a second dose containing at least about 1 μg/kg of a Factor VII polypeptide, such as at least about 10 μg/kg, such as at least about 20 μg/kg, such as at least about 40 μg/kg, such as at least about 80 μg/kg of a Factor VII polypeptide having increased activity compared to wild-type Factor VIIa ad-ministered one to 24 hours after the start of treatment.
6. Use according to embodiment 5, wherein a further, third dose containing at least about 1 μg/kg of a Factor VII polypeptide, such as at least about 10 μg/kg, such as at least about 20 μg/kg, such as at least about 40 μg/kg, such as at least about 80 μg/kg Factor VII polypeptide having increased activity compared to wild-type Factor VIIa is administered at least about one hour after the start of the second treatment.
7. Use according to any one of embodiments 1 to 6, wherein the medicament is for treatment of subjects with thrombocytopenia due to low production of platelets in the bone marrow.
8. Use according to any one of embodiments 1 to 7, wherein the medicament is for treatment of subjects with thrombocytopenia due to increased breakdown of platelets in the blood-stream and/or in the spleen and/or liver.
9. Use according to any one of embodiments 1 to 8, wherein the medicament is for treatment of subjects with thrombocytopenia due to hemodilution.
10. Use according to any one of embodiments 1 to 9, wherein the medicament is for treatment of subjects with thrombocytopenia due to specific indications selected from the list consisting of anemia, such as aplastic anemia, leukaemia, cancer in the bone marrow, infections affecting the bone marrow, alcohol-induced thrombocytopenia, immune thrombocytopenic purpura (ITP), drug-induced immune thrombocytopenia (caused e.g. by heparin), drug-induced nonimmune thrombocyopenia (caused by e.g. anticancer agents), thrombotic thrombocytopenic purpura, transfusion-induced thrombocytopenia, primary thrombocythemia, disseminated intravascular coagulation (DIC), hypersplenism (e.g. cirrhosis), hemolytic uremic syndrome, paroxysmal nocturnal hemoglobinuria, immune thrombocytopenia (such as thrombocytopenia in LED or RA), cardiopulmonary bypass, massive RBC transfusion and fluid therapy.
11. Use according to any one of embodiments 1 to 10, wherein the level of platelets is less than 150×109 platelets per liter of blood, such as less than 100×109 platelets per liter of blood, such as less than 75×109 platelets per liter of blood, such as less than 50×109 platelets per liter of blood, such as less than 40×109 platelets per liter of blood.
12. Use according to any one of embodiments 1 to 11, wherein the medicament further comprises a second coagulation agent in an amount that augments said preventing or attenuating by said Factor VII polypeptide having increased activity compared to wild-type Factor VIIa.
13. Use according to embodiment 12, wherein said second coagulation agent is selected from the group consisting of a coagulation factor and an antifibrinolytic agent.
14. Use according to embodiment 13, wherein said coagulation agent is selected from the group consisting of Factor V, Factor VIII, Factor IX, Factor X, Factor XI, Factor XIII, Fibrinogen, thrombin, TAFI, PAI-1, aprotinin, epsilon-aminocaproic acid or tranexamic acid, various antithrombotic treatments, as well as transfusions with platelet, RBC, FFP, oxygen carriers, the various bypassing agents and fluid therapies (colloids/crystalloids).
15. Use according to any one of embodiments 1 to 14, wherein said Factor VII polypeptide having increased activity compared to wild-type Factor VIIa is V158D/E296V/M298Q-FVIIa.
16. Kit of parts for treatment of bleeding episodes in a subject with thrombocytopenia, comprising
(i) A medicament comprising a Factor VII polypeptide having increased activity compared to wild-type Factor VIIa; and
(ii) Instructions for Use describing that:
a. A first dose containing at least about 1 μg/kg of a Factor VII polypeptide, such as at least about 10 μg/kg, such as at least about 20 μg/kg, such as at least about 40 μg/kg, such as at least about 80, such as at least about 100 μg/kg Factor VII polypeptide having increased activity compared to wild-type Factor VIIa, should be administered at the start of treatment;
b. Optionally, a second dose containing at least about 1 μg/kg of a Factor VII polypeptide, such as at least about 10 μg/kg, such as at least about 20 μg/kg, such as at least about 40 μg/kg, such as at least about 80 μg/kg Factor VII polypeptide having increased activity compared to wild-type Factor VIIa should be administered one to 24 hours after the start of treatment.
17. Kit according to embodiment 16, wherein the instructions for use further describes that an optional third dose containing at least about 1 μg/kg of a Factor VII polypeptide, such as at least about 10 μg/kg, such as at least about 20 μg/kg, such as at least about 40 μg/kg, such as at least about 80 μg/kg Factor VII polypeptide having increased activity compared to wild-type Factor VIIa may be administered to said subject at least about one hour after the start of the second treatment.
18. A method for treating bleeding episodes in a subject with thrombocytopenia, the method comprising administering to a subject in need of said treatment an effective amount for said treatment of a Factor VII polypeptide having increased activity compared to wild-type Factor VIIa.
19. A method according to embodiment 18, wherein the thrombocytopenia is due to low production of platelets in the bone marrow.
20. A method according to any one of embodiments 18 to 19, wherein the thrombocytopenia is due to increased breakdown of platelets in the bloodstream and/or in the spleen and/or liver.
21. A method according to any one of embodiments 18 to 20, wherein the thrombocytopenia is due to hemodilution.
22. A method according to any one of embodiments 18 to 21, wherein the medicament is for treatment of subjects with thrombocytopenia due to specific indications selected from the list consisting of anemia, such as aplastic anemia, leukaemia, cancer in the bone marrow, infections affecting the bone marrow, alcohol-induced thrombocytopenia, immune thrombocytopenic purpura (ITP), drug-induced immune thrombocytopenia (caused e.g. by heparin), drug-induced nonimmune thrombocyopenia (caused by e.g. anticancer agents), thrombotic thrombocytopenic purpura, transfusion-induced thrombocytopenia, primary thrombocythemia, disseminated intravascular coagulation (DIC), hypersplenism (e.g. cirrhosis), hemolytic uremic syndrome, paroxysmal nocturnal hemoglobinuria, immune thrombocytopenia (such as thrombocytopenia in LED or RA), cardiopulmonary bypass, massive RBC transfusion and fluid therapy.
23. A method according to any one of embodiments 18 to 22, wherein the level of platelets is less than 150×109 platelets per liter of blood, such as less than 100×109 platelets per liter of blood, such as less than 75×109 platelets per liter of blood, such as less than 50×109 platelets per liter of blood, such as less than 40×109 platelets per liter of blood.
24. A method according to any one of embodiments 18 to 23, wherein said effective amount comprises at least about 1 μg/kg of a Factor VII polypeptide, such as at least about 10 μg/kg, such as at least about 20 μg/kg, such as at least about 40 μg/kg, such as at least about 80 μg/kg, such as at least about 100 μg/kg of a Factor VII polypeptide having increased activity compared to wild-type Factor VIIa.
25. A method according to any one of embodiments 18 to 24, wherein a first amount of at least about 1 μg/kg of a Factor VII polypeptide, such as at least about 10 μg/kg, such as at least about 20 μg/kg, such as at least about 40 μg/kg, such as at least about 80 μg/kg Factor VII polypeptide having increased activity compared to wild-type Factor VIIa is administered at the start of treatment, and a second amount of at least about 1 μg/kg of a Factor VII polypeptide, such as at least about 10 μg/kg, such as at least about 20 μg/kg, such as at least about 40 μg/kg, such as at least about 80 μg/kg of Factor VII polypeptide having increased activity compared to wild-type Factor VIIa is administered to the subject one to 24 hours after the start of treatment.
26. A method according to embodiment 25, further comprising administering to the subject a third amount of at least about 1 μg/kg of a Factor VII polypeptide, such as at least about 10 μg/kg, such as at least about 20 μg/kg, such as at least about 40 μg/kg, such as at least about 80 μg/kg of Factor VII polypeptide having increased activity compared to wild-type Factor VIIa at least about one hour after the start of the second treatment.
27. A method according to any one of embodiments 18 to 26, further comprising administering to the subject a second coagulation agent in an amount that augments said treating by said Factor VII polypeptide having increased activity compared to wild-type Factor VIIa.
28. A method according to embodiment 27, wherein said second coagulation agent is selected from the group consisting of a coagulation factor and an antifibrinolytic agent.
29. A method according to embodiment 28, wherein said coagulation agent is selected from the group consisting of Factor V, Factor VIII, Factor IX, Factor X, Factor XI, Factor XIII, Fibrinogen, thrombin, TAFI, PAI-1, aprotinin, epsilon-aminocaproic acid or tranexamic acid, various antithrombotic treatments, as well as transfusions with platelet, RBC, FFP, oxygen carriers, the various bypassing agents and fluid therapies (colloids/crystalloids).
30. A method for treating bleeding episodes in a subject with thrombocytopenia in a majority of subjects with thrombocytopenia, said method comprising (i) administering to a group of subjects with thrombocytopenia having a bleeding an effective amount for said treatment of Factor VII polypeptide having increased activity compared to wild-type Factor VIIa; and (ii) observing a reduction in one or more clinical parameters of said bleeding episode among said group of subjects relative to the level of said clinical parameters that would have been expected in the same group of subjects who had not received said Factor VII polypeptide having increased activity compared to wild-type Factor VIIa.
31. A method according to any one of embodiments 18 to 30, wherein said Factor VII polypeptide having increased activity compared to wild-type Factor VIIa is V158D/E296V/M298Q-FVIIa.

1. A method for treating bleeding episodes in a subject with thrombocytopenia, the method comprising administering to a subject in need of said treatment an effective amount for said treatment of a Factor VII polypeptide having increased activity compared to wild-type Factor VIIa.

2. A method according to claim 1, wherein the thrombocytopenia is due to low production of platelets in the bone marrow, due to increased breakdown of platelets in the bloodstream and/or in the spleen and/or liver, and/or due to hemodilution.

3. A method according to claim 1, wherein the thrombocytopenia is due to a condition selected from the group consisting of anemia, leukaemia, cancer in the bone marrow, infections affecting the bone marrow, alcohol-induced thrombocytopenia, immune thrombocytopenic purpura (ITP), drug-induced immune thrombocytopenia, drug-induced nonimmune thrombocyopenia, thrombotic thrombocytopenic purpura, transfusion-induced thrombocytopenia, primary thrombocythemia, disseminated intravascular coagulation (DIC), hypersplenism, hemolytic uremic syndrome, paroxysmal nocturnal hemoglobinuria, immune thrombocytopenia, cardiopulmonary bypass, massive RBC transfusion, and fluid therapy.

4. A method for treating bleeding episodes in a subject with thrombocytopenia in a majority of subjects with thrombocytopenia, said method comprising (i) administering to a group of subjects with thrombocytopenia having a bleeding an effective amount for said treatment of Factor VII polypeptide having increased activity compared to wild-type Factor VIIa; and (ii) observing a reduction in one or more clinical parameters of said bleeding episode among said group of subjects relative to the level of said clinical parameters that would have been expected in the same group of subjects who had not received said Factor VII polypeptide having increased activity compared to wild-type Factor VIIa.

5. A method according to claim 1, wherein said Factor VII polypeptide having increased activity compared to wild-type Factor VIIa is V158D/E296V/M298Q-FVIIa.

6. A method according to claim 1, wherein said effective amount comprises not more than about 100 μg/kg of a Factor VII polypeptide.

7. A method according to claim 6, wherein said effective amount comprises not more than about 20 μg/kg of a Factor VII polypeptide.

8. A method according to claim 7, wherein said effective amount comprises not more than about 5 μg/kg of a Factor VII polypeptide.

9. A method according to claim 1, further comprising administering to the subject a second coagulation agent in an amount that augments said treating by said Factor VII polypeptide having increased activity compared to wild-type Factor VIIa.

10. A method according to claim 9, wherein said second coagulation agent is selected from the group consisting of a coagulation factor and an antifibrinolytic agent.

11. A method according to claim 10, wherein said coagulation agent is selected from the group consisting of Factor V, Factor VIII, Factor IX, Factor X, Factor XI, Factor XIII, Fibrinogen, thrombin, TAFI, PAI-1, aprotinin, epsilon-aminocaproic acid, tranexamic acid, an antithrombotic treatment, and transfusions with one or more of platelet, RBC, FFP, and oxygen carriers.

12. A kit of parts for treatment of bleeding episodes in a subject with thrombocytopenia, comprising
(i) A medicament comprising a Factor VII polypeptide having increased activity compared to wild-type Factor VIIa; and
(ii) Instructions for Use describing that:
a. A first dose containing no more than about 100 μg/kg of a Factor VII polypeptide having increased activity compared to wild-type Factor VIIa, should be administered at the start of treatment;
b. Optionally, a second dose containing no more than about 100 μg/kg Factor VII polypeptide having increased activity compared to wild-type Factor VIIa should be administered one to 24 hours after the start of treatment.


SEQUENCE LISTING (1 sequence)

SEQ ID NO: 1 - protein, 406 aa, Homo sapiens [sequence omitted]